(19) United States
(12) Patent Application Publication
(10) Pub. No.: US 2010/0178330 A1
(43) Pub. Date: Jul. 15, 2010

(54) ASYMMETRIC LIPOSOMES AND USES IN MEDICAL FIELD THEREOF

(76) Inventors: Maurizio Fraziano, Roma (IT); Gianluca Quintiliani, Roma (IT); Emanuela Greco, Roma (IT); Marco De Spirito, Roma (IT)

Correspondence Address: NEIFELD IP LAW, PC, 4813-B EISENHOWER AVENUE, ALEXANDRIA, VA, 22304, US

(21) Appl. No.: 12/667,257

(22) PCT Filed: Jul. 15, 2008
(86) PCT No.: PCT/IT08/00474

§ 371 Date: Dec. 30, 2009

(30) Foreign Application Priority Data
Jul. 16, 2007 (IT) RM2007A000394

(51) Int. Cl. A61K 9/127; A61K 31/66; A61K 9/12; A61P 31/04; A61P 31/12

(52) U.S. Cl. 424/450; 514/114

(57) ABSTRACT

The invention concerns new asymmetric liposomes and uses thereof in medical field to transport lipids involved in antimicrobial or antiviral response, particularly at level of pulmonary target cells.

The present invention concerns new asymmetric liposomes and uses in medical field thereof to transport lipids involved in antibacterial and/or antiviral response, in particular at level of pulmonary target cells.

The pathogenicity of many intracellular bacteria (i.e. Mycobacterium tuberculosis, Pseudomonas aeruginosa, Streptococcus pneumoniae, Klebsiella pneumoniae) is based on the ability in persisting and replicating within the cellular environment after phagocytosis thereof (i.e alveolar macrophages).

The phagocytosis process involves the extension of phagocyte plasmatic membrane around the recognised microbe, joining of two extremities and their bonding: the foreign particle thus is incorporated inside of a membrane bound vesicle, the endosome, that detaching from the plasmatic membrane inside of the cytoplasm, forms the phagosome. On the separation the composition of particle enveloping membrane is the same as plasmatic one (Muller W. A et al. J. Cell. Biol. 1983; 96:29-36), but few minute later it acquires several receptors among which mannose receptor (MMR) and Rab5, a small GTPase that plays an important role during the first steps of phagosome maturation process (Viera V. et al. Biochem J. 2002; 366; 689-704). Subsequently the phagosomes are fused with lysosomes, resulting in the so-called phagolysosomes, wherein the degradation of phagocytated material occurs with subsequent expulsion thereof from the cell by exocytosis. Adhesion, ingestion, phagosome formation and phagolysosome maturation steps involve the phagocyte activation, with increase of cellular metabolism. Phagolysosome during maturation loses Rab5 and MMRcs and incorporates:
Rab7, small GTPase regulating phagosome lysosome fusion;
Lysosomes associated membrane proteins, (LAMP);
Lysosomal acid protease, cathepsin D (Desjardins M. Trends Cell Biol. 1995; 5; 183-186).
Over the time not only receptor type but also receptor number are variable: in fact LAMP and cathepsin D progressive increase are observed (Clemens D. L et al. J. Exp. Med. 1995; 181; 257-270).
Macrophage activated anti-microbial systems, after the phagocytosis, can follow two pathways: i.e oxygen-dependent and oxygen-independent pathways. As to the oxygen-dependent pathway, on the external surface of plasmatic membrane an oxidase enzyme reduces molecular oxygen (O₂) to superoxide (O₂⁻) anion, that within the cell is transformed to hydrogen peroxide (H₂O₂) by superoxide dismutase enzyme. Hydrogen peroxide within phagolysosome is transformed by myeloperoxidase, in the presence of halogens, to hypoclorite ion (ClO⁻) having strong bactericidal activity. Oxygen-independent antibacterial system acts mostly during the anaerobic bacteria killing resulting in lower pH within phagolysosome. This system includes like antibacterial agents cationic proteins of primary granules, i.e. lactoferrin, suitable to bind Fe, and lysozyme. The bactericidal effect can be mediated also by nitrogen active intermediates (RNI) and nitric oxide (NO) generated by L-arginine substrate through the activity of inducible nitric oxide synthase enzyme (iNOS). All these microbicidal substances essentially are produced inside the lysosomes and phagolysosomes, wherein they act on incorporated microbes without phagocytes damage.

Above described inhibition of phagolysosome maturation is a pathogenetic mechanism often used by many pathogenic bacteria in order to deal with the macrophage antimicrobial response. The phagolysosome biogenesis is regulated by various enzymes (Vergne I. et al., Annu. Rev. Cell. Dev. Biol. 2004; 20:367-94) and second lipid messengers among which Sphingosine, Sphingosine 1-phosphate (S1P), lysophosphatidic acid (LPA), phosphatidic acid (PA), sphingomyelin (SM), ceramide (Cer), arachidonic acid (AA) (Anes and et al., Nat. Cell. Biol. 2003; 5:793-802).

Frequently, pulmonary infections are associated with inflammatory response that, if on the one hand acts in order to control the infection, from the other hand generates tissue damages whose extent is proportional to the microorganism ability to escape from the cell-mediated immune response.

Mycobacterium tuberculosis (MTB) is a pathogen preferentially using as an host alveolar macrophage due to entrance facilitating entrance surface receptors (Daeron M. Annu. Rev. Immunol. 1997; 15:203-234).

In this particular context of M. tuberculosis infection Armstrong and Hart (Armstrong J. A et al. J. Exp. Med. 1975; 142:1-16) more than 30 years ago showed that M. tuberculosis containing phagosomes do not complete their maturation step and therefore the inhibition of the phagolysosome maturation represents one of the key mechanisms through which these pathogens are able to survive within macrophages.

Phagosomes containing viable and virulent mycobacteria express molecules as transferrin receptor, class II MHC molecules and GM1 ganglioside, typical for an initial phagosome maturation step meanwhile lack those molecules typical of late maturation step, like mannose receptor, lysosome protease Cathepsin D and membrane H⁺-ATPases (Clemens D. L et al. J. Exp. Med. 1995; 181; 257-270). It is supposed further that the absence of H⁺-ATPase is the cause of the reduced acidification of phagosomes (Sturgill-Koszycki S. et al. Science. 1994; 263:678-81) containing mycobacteria, whose pH is maintained from 6.2 to 6.3, instead to reach 5.3-5.4 values normally found in endosome environments.

The presence of M. tuberculosis within phagosome induces a reduction of LAMP, cathepsin D expression and Rab5 persistence (Clemens D. L et al. J. Exp. Med. 181; 257-270. 1995). The presence of Rab5 but not of Rab7 in mycobacterium phagosome is indicative of the phagosome maturation arrest (Deretic V. et al. the Mol Microbiol. 1999; 31:1603-1609).

From recent studies an important role of a macrophage enzyme, i.e. D phospholipase (PLD), in anti-M. tuberculosis response has been found. This enzyme appears to be been involved, in fact, in the phagolysosome maturation processes, being therefore a crucial step in microbicidal mechanisms and important member in the anti-mycobacterial innate response (Kusner D. J et al. J. Immunol. 2000; 164; 379-388).

Macrophages, after a microorganism phagocytosis, respond with an increase of intracellular Ca⁺⁺ concentration from a basal level of 50-100 nM to 500-1000 nM (Malik Z. A et al. J. Exp. Med. 191; 287-303. 2000). While cytosolic Ca⁺⁺ increase is not involved in the phagocytosis (DiVirgilio F. et al. J. Cell Biol. 1988; 106:657-666), it becomes fundamental in effector mechanisms of innate immune system, as in generation of oxygen reactive intermediates (Korchak H. M et al. J. Biol. Chem. 1988; 263; 11090-11097), and phagolysosome maturation (Worth R.RPM et al. Proc. Natl. Acad. Ski. USA. 2003; 100; 4533-4538). While in living M. tuberculosis infected macrophages, Ca⁺⁺ concentration increase is not detected, the phagocytosis of heat killed M. tuberculosis results in cytosolic Ca⁺⁺ increase and maturation of phagosomes to phagolysosomes. Various evidences demonstrate that the inhibition of Ca⁺⁺ increase in macrophages is fundamental for tuberculosis pathogenesis; in fact it has been demonstrated that, during M. tuberculosis phagocytosis, an increase of the drug induced cytosolic Ca⁺⁺, favours the phagosome maturation and a better intracellular killing of tubercular bacilli (Malik Z. A et al. J. Immunol. 2001; 166:3392-3401). During the killed MTB phagocytosis, the increase of cytosolic Ca⁺⁺ results from the activation of a macrophage enzyme, i.e. sphingosine kinase (Malik Z. A et al. J. Immunol. 2003; 170; 2811-2815), which catalyses the conversion from sphingosine to sphingosine 1-phosphate, a bioactive lipid stimulating the release of the Ca⁺⁺ from intracellular reservoirs (Spiegel S. et al. J. Biol. Chem. 2002; 277; 25851-25854). Moreover the activation of sphingosine kinase involves translocation thereof from cytoplasm to phagosome formation region. On the contrary living M. tuberculosis inhibits the activation of the sphingosine kinase and translocation thereof into mycobacterium containing phagosome (Kusner D. J. Clin Immunol. 2005; 114:239-247). These results suggest that sphingosine kinase represents, in tuberculosis pathogenesis, a molecular target, whose inhibition by M. tuberculosis results in arrest of phagosome maturation and macrophage bactericidal activity.

Currently used therapies for tuberculosis treatment involve the administration of various antibiotics divided in two groups based on the occurrence of possible resistance. The first group consists of isoniazid, (INH), rifampicin, pyrazinamide, and ethambutol and generally suggested as first-line therapy due to effectiveness and minor toxicological profile thereof (Gilman; A. RPM. In The Pharmacologic Basis of Therapeutics; A. G. Gilman, Ed; Pergamon Press: New York, 1990; pp. 1061-1162). The therapy involve daily administration of four antibiotics concurrently during first two months (intensive period) and NIH and rifampicin during following four months (follow-up period). The strategy underlying this therapeutic regimen is to eliminate the first step actively proliferating and residual bacilli, in order to prevent endogenous re-infections and pharmacological resistances, in the follow-up period.

The second group of antibiotics, very rarely used, except in the geographic areas with drug-resistances, includes, but it is not limited to fluorochinolones (ofloaxacin, ciprofoxacin), aminoglycosides, cycloserine, macrolides, ethionamide, para-aminosalycilic acid (PAS), thiacetazone.

The occurrence of multi-drug resistant mycobacterial strains represents, today, one among the greater impediments for an effective pharmacological treatment of TB.

It is apparent that the continuous occurrence of new drug-resistant strains, the long lasting antibiotic therapy often interfering with anti-troviral drugs (Niemi M. et al. Clin Pharmacokinet. 2003; 42:819-50), the need to kill also quiescent intracellular bacteria results in a need of a pharmacological approach suitable to ideally enhance directly the anti-mycobacterial macrophage response meanwhile the pathogenetic inflammatory response is maintained under control.

This requirement, particularly important for tubercular infection, is found in all other intracellular infections by bacterial or viral agents wherein it is necessary to enhance the macrophage microbicidal response (or other cell types like fibroblasts, epithelial or endothelial cells) at pulmonary level. These pulmonary infections are often associated to inflammatory response that, if on one hand aims to control the infection, from the other hand generates tissue damages whose extent is proportional to the microorganism ability to escape from the cell-mediated immune response. Consequently the capability to increase the cellular microbicidal activity associated to concurrent decrease of the tissue damaging inflammatory response could be therapeutically important.

The authors of the present invention now have found that, using apoptotic body phagocytosis as entrance pathway it is possible to transport directly into the target cell second lipid messengers suitable to enhance or restore the antiviral/antibacterial response of the host, concurrently decreasing tissue damaging inflammatory response, by means of an asymmetric liposome system designed to mimic said apoptotic bodies. The approach is based generally on the innate immune system enhancement representing first-line defence against foreign microbial attacks. Since such defences are intrinsically non-specific, enhancement thereof allows a more effective response against a broad pathogen type to be exerted.

More specifically the authors have explored the possibility to enhance/restore the macrophage microbicidal response (or other cellular types) by transporting directly into host cell lipid intermediates (fatty acids, phospholipids, etc.), known to be involved in antibacterial (i.e biogenesis of phagolysosome) or antiviral response through the generation of asymmetric liposomes characterised by the presence of phosphatidylserine and bioactive lipid outside and inside, respectively. The outside phosphatidylserine presence allows an efficient phagocytosis not only by macrophages but also those cell types suitable to phagocitate apoptotic bodies expressing outside phosphatidylserine, like fibroblasts, epithelial and endothelial cells which represent possible target cells for viral and bacterial pulmonary infections (i.e. Mycobacterium sp; Streptococcus pneumoniae, Klebsiella pneumoniae; Pseudomonas aeruginosa; Enterobacter sp.; Fusobacterium nucleatum; Bacteroides melaminogenicus; Haemophilus influenzae; Legionella sp.; influenza and para-influenza virus; syncytial respiratory virus; coronavirus; adenovirus). Moreover, phagocytosis through recognition of phosphatidylserine molecules presents further advantage of being associated to the production of anti-inflammatory cytokines and to reduce the intensity of the antigen-specific in vivo response (Hoffmann P. R et al. J. Immunol. 2005; 174:1393-1404), thus reducing the tissue damaging inflammatory response.

In scientific literature symmetrical liposomes consisting also of phosphatidylserine (both in inside and outside lipid layer) are known and are used to transport protein antigens, DNA, both large and small size drugs for vaccine and therapeutic purpose (Gregoriadis RPM. Trends Biotechnol. 1995; 13: 527-37). Therefore up to now the association of phosphatidylserine with bioactive lipids (second lipid messengers), asymmetrically distributed along the liposome membrane and involved in the activation of antibacterial or antiviral pathways has not been described. Further it is apparent that literature cited liposomes, suitable to transport hydrophilic or hydrophilic moiety containing molecules would not be suitable for an efficient lipid encapsulation since their inside environment is a water or saline buffer based liquid wherein a lipid difficulty can be encapsulated.

The asymmetric liposomes according to the present invention couple the necessity of using lipids in order to build up the liposome scaffold with that of using lipids asymmetrically disposed on both surfaces of liposome membrane. This characteristic allows some problems often occurring during the encapsulation process of the molecule to be transported, which is often chemically modified to increase the encapsulation efficiency, to be overcome.

The therapeutic approach according to the present invention is applicable according to a specific embodiment thereof to Mycobacterium sp. (M. tuberculosis; M. bovis; M. africanum; M. lepre; M. ulcerans) whose pathogenetic mechanism interferes with the host (human or animal) antimicrobial response, by inhibition of phagolysosome maturation inside of the macrophages.

With particular reference to the tubercular infection, a pharmacological approach of this type to be adopted in medical or veterinary field could be strategically associated to conventional antibiotic therapy in order i) to attack the pathogen from various points of view, ii) to decrease, like in the case of the TB, the long therapy periods and iii) to prevent the occurrence of antibiotic resistant bacterial strains thus overcoming some of the limits of the known art.

As mentioned above the asymmetric liposome system that mimics apoptotic bodies can represent a technological base in order to enhance the response against intracellular bacterial and viral pathogens generating infections, mainly at pulmonary level. The lung is preferred therapeutic target because i) it is particularly susceptible to bacterial and viral infections; ii) the bacterial and viral pneumonias are a major cause of morbility and mortality among aged and immuno-depressed people; iii) liposomes and/or micro-/nano-particles for drug transport are suitable to aerosol administration (Zahoor A et al. Int. J. Antimicrob. Agents. 2005; 26:298-303; Vyas S P et al. Int. J. Pharm. 2004; 269:37-49). This administration mode, in the case of pulmonary infections, displays various advantages over the systemic one: i) directed administration of the drug into the target organ, ii) reduction of systemic side-effects, iii) extension of drug mean life time in the interest organ.

In any case if other possible regions as derma or intestinal mucosa are considered, it is possible to use liposome formulations for topical or oral administration, in the form of gel or cream or having gastric environment protective components.

Therefore it is an object of the present invention asymmetric liposomes or aggregates thereof characterised in that they are mimetic apoptotic bodies and comprise phosphatidylserine molecules within the external lipid layer and at least one bioactive lipid involved in antibacterial and/or antiviral response inside thereof. Said at least one bioactive lipid(s) is(are) suitable to restore and/or enhance the correct cellular antibacterial antiviral pathogen-related response and escape mechanism of the antimicrobial response triggered by the latter. Preferably, said lipids are selected from the group consisting of phosphatidic acid, lysophosphatidic acid, arachidonic acid, sphingomyelin, sphingosine, sphingosine 1-phosphate, ceramide, leukotrienes, prostanoids, cyclopentenone prostaglandins (i.e. PGA1, PGA2, PGJ2) and possible derivatives thereof, but such selection is not to be considered in a limitative way. According to a particularly preferred embodiment of the present invention said liposomes comprise inside as bioactive lipid phosphatidic acid or derivatives thereof. Alternatively, said liposomes comprise as bioactive lipid inside a cyclopentenone prostaglandin selected from PGA1, PGA2, PGJ2 or derivatives thereof. According to an alternative embodiment it is possible to include more than one bioactive lipids inside of a “polyvalent” named liposome; i.e at least one cyclopentenone prostaglandin selected from PGA1, PGA2, PGJ2 and phosphatidic acid.

The present invention refers specifically to asymmetric liposomes or aggregates thereof as above described, for use in medical field, in particular for the treatment of pulmonary infections deriving from intracellular bacterial and/or viral pathogens. In fact, it can occurs that a viral infection (HIV virus type) results in an immuno-suppressed condition such that it can make the host particularly susceptible to a bacterial infection (Mycobacterium tuberculosis type). In this context, the tubercular, and HIV virus infections results in parallel mutually reinforcing epidemics. In fact, if from one hand HIV infection makes the host more susceptible to the tubercular disease development on the other hand the latter favours the HIV replication, increasing by 10-30 times the viral infection progression rate towards AIDS established conditions. Currently, approximately a third of 42 million HIV/AIDS affected persons are affected also by Tuberculosis

A further object of the present invention is the use of asymmetric liposomes or aggregates thereof as above defined for the preparation of a medicament for the treatment of pulmonary infections deriving from intracellular bacterial pathogens selected from Mycobacteria sp. (preferably Mycobacterium tuberculosis, Mycobacterium bovis, Mycobacterium africanum, Mycobacterium avium, Mycobacterium ulcerans, Mycobacterium leprae); Streptococcus pneumoniae; Klebsiella pneumoniae; Pseudomonas aeruginosa; Enterobacter sp.; Fusobacterium nucleatum; Bacteroides melaminogenicus; Haemophilus influenzae and Legionella sp. or from intracellular viral pathogens selected from influenza and para-influenza virus, respiratory syncytial virus; coronavirus; adenovirus and HIV.

According to an alternative embodiment the present invention refers to the specific use of asymmetric liposomes characterised in that they are mimetic of apoptotic bodies and comprise phosphatidylserine molecules within the external lipid layer and phosphatidic acid inside, for the preparation of a medicament for the treatment of tuberculosis or HIV infection associated tuberculosis. In this case it is possible to provide the administration of single liposome comprising, in addition to phosphatidic acid as bioactive lipid, also at least one cyclopentenone prostaglandin selected from PGA1, PGA2, PGJ2 or derivatives thereof; alternatively it is possible to provide a sequential, separate or simultaneous administration of separated liposomes comprising as bioactive lipids phosphatidic acid and at least one cyclopentenone prostaglandin selected from PGA1, PGA2, PGJ2 or derivatives thereof, respectively.

The present invention further concerns a pharmacological combination of active principles comprising one or more asymmetric liposomes according to the invention. Preferably said more than one asymmetric liposomes in the pharmacological combination are separated liposomes comprising as bioactive lipids phosphatidic acid and at least one cyclopentenone prostaglandin selected from PGA1, PGA2, PGJ2, respectively.

The invention further concerns a pharmacological combination of active principles comprising one or more asymmetric liposomes according to the invention and at least an antibiotic or an antiviral (i.e antiretroviral; anti-HIV). According to a preferred embodiment the present invention concerns a pharmacological combination comprising asymmetric liposomes characterised in that they comprise phosphatidylserine molecules within the external lipid layer and phosphatidic acid inside and at least an antibiotic selected from first-line (isoniazid, rifampicin, pyrazinamide, ethambutol, streptomycin) and second-line anti-tubercular drugs (fluorochinolones, aminoglycosides, cycloserine, macrolides, ethionamide, para-aminosalycilic acid (PAS), thiacetazone.

According to a particularly preferred embodiment the invention refers to a pharmacological combination of this type (in association with an antibiotic or antiviral) wherein said asymmetric liposomes comprise in addition to phosphatidic acid as bioactive lipid at least one cyclopentenone prostaglandin selected from PGA1, PGA2, PGJ2; alternatively it is possible to provide the administration of more than one asymmetric liposomes, i.e comprising as bioactive lipids phosphatidic acid and at least one cyclopentenone prostaglandin selected from PGA1, PGA2, PGJ2, respectively. This type of combination administration can be useful, for example, for treatment of tuberculosis in association with HIV infection.

According to a preferred embodiment the present invention concerns a kit of parts comprising one or more asymmetric liposomes according to the invention for simultaneous, separated or sequential use for the therapy of pulmonary infections. Preferably said more than one asymmetric liposomes comprise inside thereof as bioactive lipid phosphatidic acid and at least one cyclopentenone prostaglandin selected from PGA1, PGA2, PGJ2, respectively, for simultaneous, separated or sequential use for the therapy of tuberculosis, in particular when HIV infection associated.

According to a further aspect the invention refers to a kit of parts comprising one or more asymmetric liposomes according to the invention as above defined and at least an antibiotic and/or an antiviral, for simultaneous, separated or sequential use for the therapy of pulmonary infections. Preferably, said asymmetric liposomes are characterised in that they comprise phosphatidylserine molecules within the external lipid layer and phosphatidic acid inside and said antibiotic is selected from first-line (isoniazid, rifampicin, pyrazinamide, ethambutol) and second-line anti-tubercular drugs (fluorochinolones, aminoglycosides, cycloserine, macrolides, ethionamide, para-aminosalycilic acid (PAS), thiacetazone) for simultaneous, separated or sequential use for the therapy of tuberculosis.

According to a particularly preferred embodiment of above defined kit of parts, said more than one asymmetric liposomes comprise separated liposomes comprising as bioactive lipids inside thereof phosphatidic acid and at least one cyclopentenone prostaglandin selected from PGA1, PGA2, PGJ2, for simultaneous, separated or sequential use for the therapy of the tuberculosis, in particular HIV associated tuberculosis.

According to a further embodiment of kit of parts said asymmetric liposomes comprise as bioactive lipids inside thereof phosphatidic acid and at least one cyclopentenone prostaglandin selected from PGA1, PGA2, PGJ2, for the simultaneous, separated or sequential use for the therapy of the tuberculosis, HIV associated tuberculosis.

A pharmaceutical composition comprising the asymmetric liposomes as above defined as active principle together with one or more pharmacologically acceptable excipients and/or adjuvants, constitutes a further object of the present invention suitable to the administration by aerial, dermal or mucosal (i.e intestinal mucosa) mode.

The present invention further refers to a pharmaceutical composition comprising the pharmaceutical combination as above defined, together with to one or more pharmacologically acceptable adjuvants and/or excipients.

According to a preferred embodiment of the present invention the pharmaceutical composition suitable to the administration by aerial mode is formulated as an aerosol; the use of liposome and/or micro/nano-particle to transport drugs is well suitable to aerosol administration mode. As to this, as it is known to those skilled in the art, in order to transport liposomes by means of aimed aerosol therapy, that is targeted to specific pulmonary sections, it is necessary the produced liposomes size to be modulated. In fact, various studies demonstrated that the best therapeutic effectiveness is obtained using particulates having sizes from 100 nm to 50 mm (depending on selected lung area for the therapy). A possible way in order to overcome this limitation consists of producing stable aggregates comprising two or more liposomes, thus modulating the particulate size. Currently the authors of present the invention are about to optimise a procedure for production of liposome aggregates based on known methodical (Edges F. et al. Langmuir 5214, 22 2004) allowing loaded liposomes to be assembled (i.e with polar heads).

The invention has as a further object the use of above defined pharmaceutical composition(s) for the preparation of a medicament for the treatment of pulmonary infections deriving from intracellular bacterial, pathogens selected from Mycobacteria sp. (preferably Mycobacterium tuberculosis, Mycobacterium bovis, Mycobacterium africanum, Mycobacterium avium, Mycobacterium ulcerans, Mycobacterium leprae); Streptococcus pneumoniae; Klebsiella pneumoniae; Pseudomonas aeruginosa; Enterobacter sp.; Fusobacterium nucleatum; Bacteroides melaminogenicus; Haemophilus influenzae; Legionella sp. or viral pathogens selected from influenza and para-influenza virus, respiratory syncytial virus; coronavirus; adenovirus and retrovirus (HIV).

A further object of the present invention is a carrier system comprising at least one liposome or micro- or nano-particle or aggregated thereof, characterised in that they are asymmetric and mimic the apoptotic bodies and comprise phosphatidylserine molecules within the external lipid layer and at least one bioactive lipid inside thereof to transport said at least a lipid to cells suitable to phagocitate apoptotic bodies. Said at least one bioactive lipid is lipid(s) suitable to restore and/or to enhance the correct cellular antibacterial and/or antiviral response related to the pathogen and escape mechanism of the antimicrobial response triggered by the latter. According to of the present invention the term “antimicrobial response” means indifferently a cellular defence response against a bacterial or viral agent; the term “antibacterial response” means cellular defence response against a bacterial agent; the term “antiviral response” means a cellular defence response against a viral agent. Preferably, said at least one lipid is selected from the group consisting of phosphatidic acid, lysophosphatidic acid, arachidonic acid, sphingomyelin, sphingosine, sphingosine 1-phosphate, ceramide, leukotrienes, prostanoids, cyclopentenone prostaglandins (i.e. PGA1, PGA2, PGJ2) and all derivatives thereof, but such selection is not to be considered in a limitative way. The carrier system object of the present invention preferably is designed for pulmonary cells suitable to phagocitate apoptotic bodies selected from phagocytes, macrophages (preferably alveolar), fibroblasts, epithelial and endothelial cells. In any case it is possible to direct the carrier system also to other cellular targets in addition to pulmonary as dermal or mucosal cells (i.e intestinal mucosa). It will be appropriate to formulate adequately such carrier system, for example using liposome formulations in the form of gel or cream in the case of dermal application. Analogously for mucosal (for example intestinal) administration it will be appropriate these liposome formulations to be protected with protective components against gastric environment.

According to a particularly preferred embodiment said at least one bioactive lipid contained inside of the carrier system is phosphatidic acid or derivatives thereof and said target cells are macrophages, preferably alveolar, or epithelial alveolar cells. According to another particularly preferred embodiment said at least one bioactive lipid contained inside of the carrier system is a cyclopentenone prostaglandin selected from PGA1, PGA2, PGJ2 or derivatives thereof and said target cells are macrophages, preferably alveolar, epithelial alveolar cells, fibroblasts and endothelial cells. According to an alternative embodiment it is possible to incorporate inside of one or more bioactive lipids a “polyvalent” named liposome i.e. at least one cyclopentenone prostaglandin selected from PGA1, PGA2, PGJ2 and phosphatidic acid or derivatives thereof. Preferably, said carrier system is characterised in that it is formulated as an aerosol for aerial administration.

Finally the invention refers to a method to transfer bioactive lipids to cells suitable to phagocitate apoptotic bodies which involves the administration of at least one carrier system as above defined. Said cells suitable to phagocitate apoptotic bodies are preferably pulmonary, dermal or mucosal, preferably intestinal, cells. More preferably, said pulmonary cells are selected from phagocyte, macrophages (preferably alveolar), fibroblasts, epithelial cells, endothelial cells. The asymmetric composition of these liposomes in fact has been designed in order to secure, through the presence of phosphatidylserine outside, an efficient phagocytosis not only by macrophages and phagocytes but also by all cell types suitable to phagocitate apoptotic bodies, as fibroblasts, epithelial cells, endothelial cells, and described as potential target cells for bacterial and viral pulmonary infections.

According to a preferred embodiment of the present invention the above reported method involves the administration of the carrier system by an aerosol mode.

The present invention now will described by an illustrative, but not limitative way, according to preferred embodiments thereof with particular reference to the enclosed drawings, wherein:

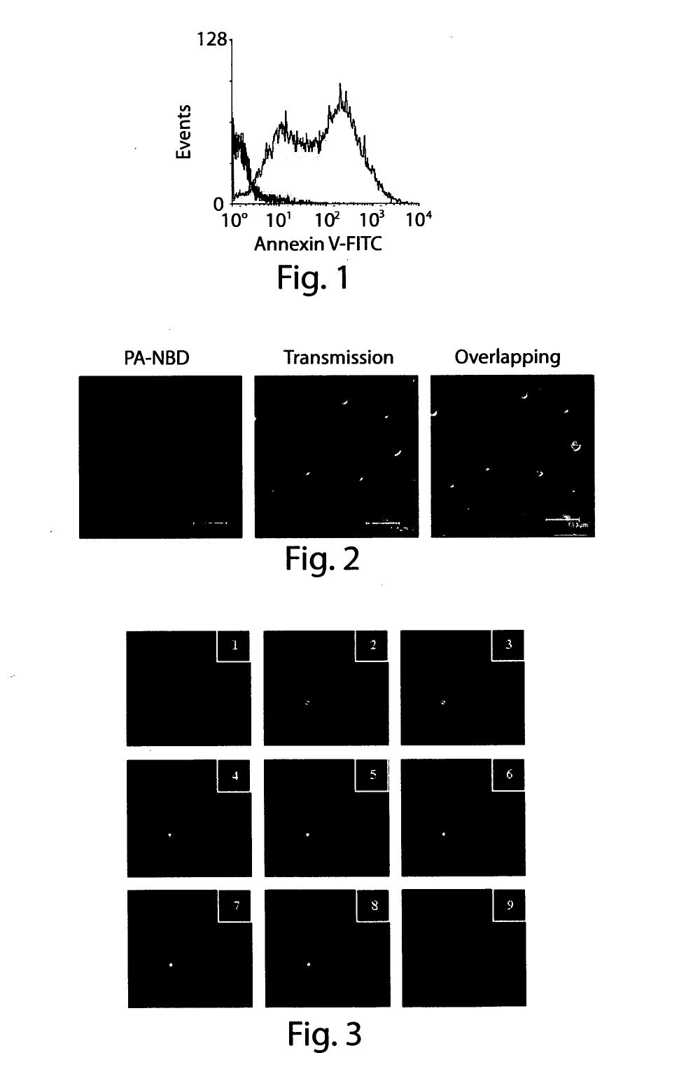

FIG. 1, shows the cytofluorimetric characterisation of asymmetric liposomes containing phosphatidylserine outside. The liposomes containing phosphatidylserine outside and phosphatidic acid inside (PS/PA) thereof have been labelled with FITC conjugated Annexin V and analysed by cytofluorimetry, by overlapping of unlabelled liposome auto-fluorescence to Annexin V-FITC labelled liposome fluorescence;

FIG. 2 shows liposomes with phosphatidylserine outside and fluorescent phosphatidic acid inside (PS/PA_{NBD}), having various sizes, observed by confocal fluorescence microscopy;

FIG. 3 shows the phagocytosis of the liposome containing phosphatidylserine outside and fluorescent phosphatidic acid inside (PS/PA_{NBD}) through various sagittal cell planes scanning (panels 1-9); THP-1 monocytoid cell line have been induced to differentiate in macrophages (dTHP-1) and exposed to liposomes consisting of phosphatidylserine outside and fluorescent phosphatidic acid inside (PS/PA_{NBD}) and various sagittal plane scanned using fluorescence microscopy.

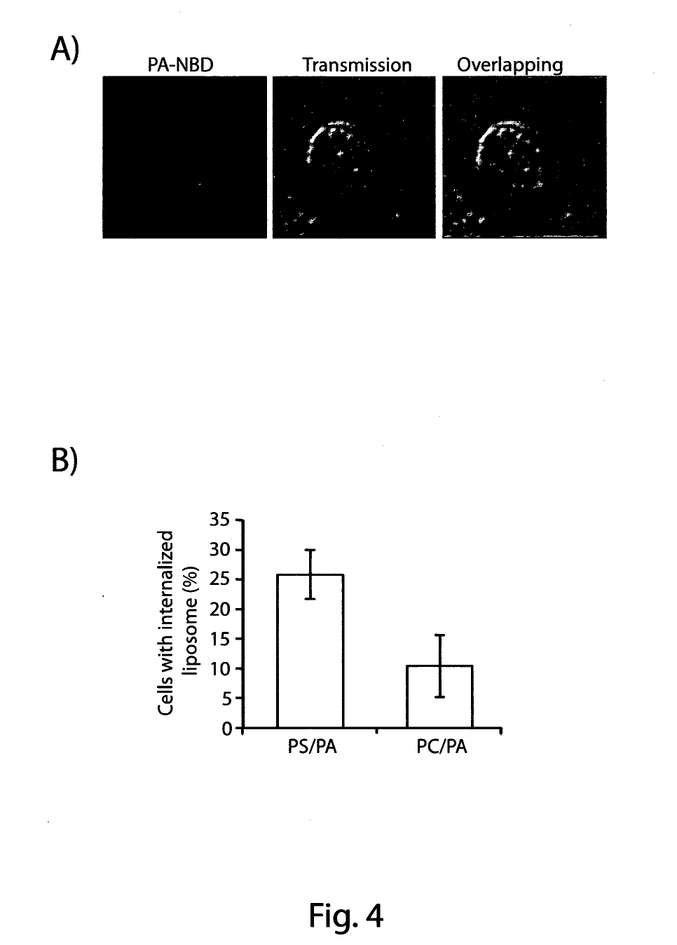

FIG. 4 shows intracellular localisation of liposome containing phosphatidylserine outside and phosphatidic acid inside; panel A shows the analysis using confocal fluorescence microscopy of dTHP-1 cells exposed to liposomes consisting of phosphatidylserine outside and fluorescent phosphatidic acid inside (PS/PA_{NBD}) (green); panel B shows dTHP-1 cells exposed to PS/PA_{NBD} or PC/PA_{NED}; the analysis of cells containing internalised liposomes with the fluorescent PA with respect to total number of cells using confocal fluorescence microscopy was carried out after incubation for 90 minutes, counting at least 100 cells for each sample; data is shown as average±DS of % cells containing at least one internalised liposome with respect to total cells obtained in 3 independent experiments;

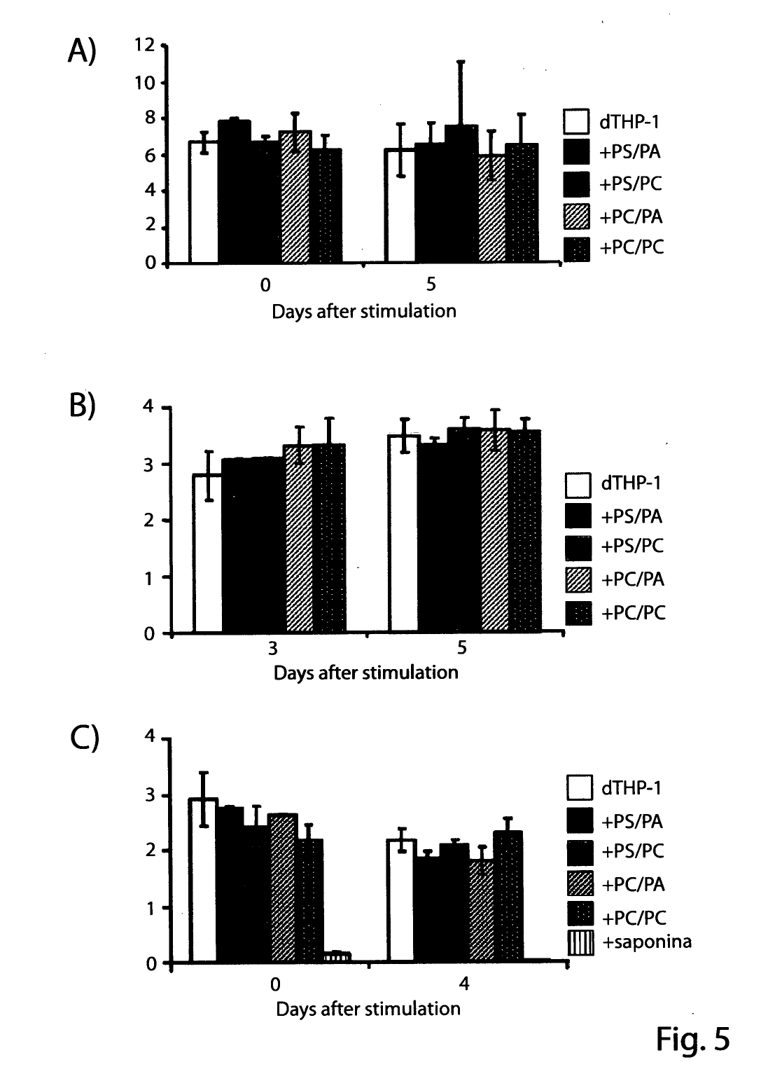

FIG. 5 shows the citotoxicity analysis of the various liposome preparations; the cells have been exposed to following liposome preparations: i) liposome containing phosphatidylserine outside and phosphatidic acid (PS/PA) inside, ii) liposome containing phosphatidylserine outside and phosphatidylcholine inside (PS/PC); iii) liposome containing phosphatidylcholine outside and phosphatidic acid inside (PC/PA); iv) liposome containing phosphatidylcholine both outside and inside (PC/PC). At time points reported in Figure i) a counting of the cell viability using Trypan Blue exclusion method was carried out (panel A), ii) release of Lactate Dehydrogenates in cell supernatant was monitored (panel B) and iii) the formation of formazan crystal from 3-(4,5-dimethyl thiazol-2yl)-2,5-diphenyl tetrazolium bromide (MTT) (panel C); results shows the mean±DS of values obtained from 3 independent experiments;

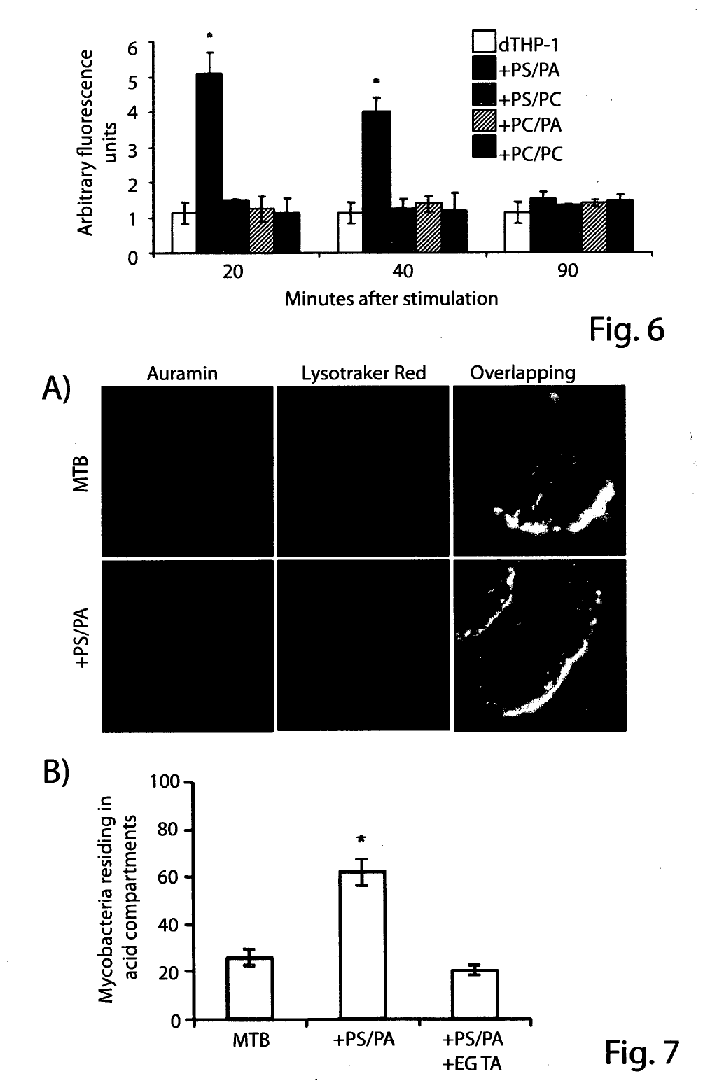

FIG. 6 shows the increase of intracellular Ca²⁺ levels as a result of the stimulation with PS/PA liposomes; THP-1 cells have been stimulated with liposomes of various type (PS/PA; PS/PC; PC/PA; PC/PC); the levels of intracellular Ca²⁺ have been evaluated as fluorescence arbitrary units at 20, 40 and 90 minutes after stimulation; not stimulated cells represent negative control; results are shown as mean±DS of the values obtained from triplicate of each condition; *p=0.006 vs not treated control;

FIG. 7 shows localisation of M. tuberculosis in endosome acid compartments, after stimulation with PS/PA liposomes; panel A shows cells treated with Lysotracker red (red) to visualise acid compartments and subsequently infected with M. tuberculosis; cells have been fixed and then auramin (green) stained to label mycobacteria; panel B shows the quantitative analysis as mean percentage±DS of mycobacteria present in acid compartments with respect to the total of the intracellular bacteria; the analysis has been carried out in 3 independent experiments, considering at least 30 bacilli for sample; *p<0.001 compared to not treated control;

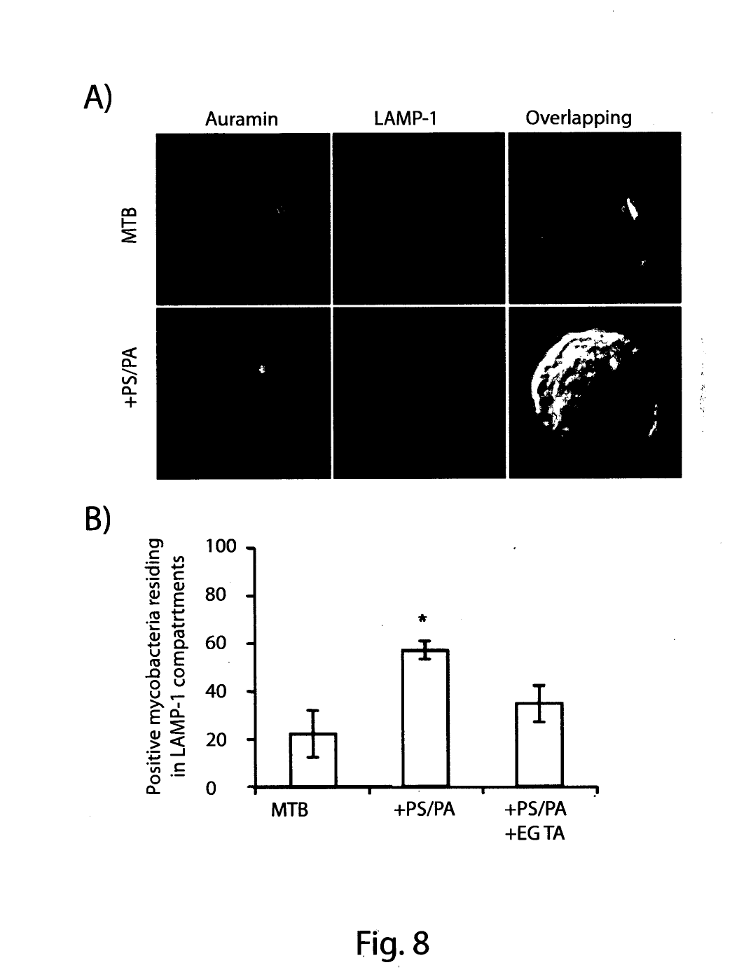

FIG. 8 shows localisation of Mycobacterium tuberculosis in late phagolysosomes I after stimulation with PS/PA liposomes; the panel depicts the visualisation of late phagolysosome compartments by labelled cells with anti-LAMP-1 monoclonal antibody (red) and mycobacteria by means of fixation and staining of the cells with auramin (green); panel B shows the quantitative analysis as mean percentage±DS of mycobacteria present in LAMP-1 expressing compartments, with respect to total of intracellular bacteria; the analysis has been carried out in 3 different experiments, considering at least 30 bacilli for sample; *p<0.001; compared to not treated control.

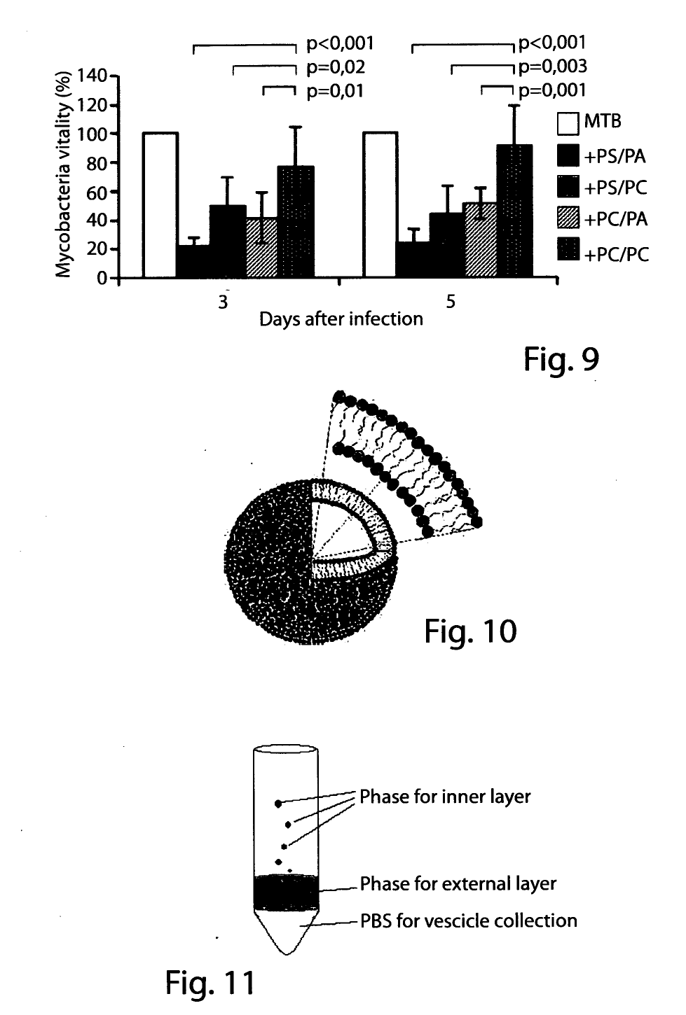

FIG. 9 shows the intracellular viability of M. tuberculosis in cells stimulated with different liposome types; cells have been infected with M. tuberculosis at MOI of 1 bacterium/cell and stimulated with PS/PA, PS/PC, PC/PA PC/PC liposomes in triplicate; at indicated time points and on each culture well the CFU assay has been carried out in triplicate; data is expressed as mean±DS of all values obtained compared to negative control represented by MTB infected macrophages;

FIG. 10 depicts the double lipid layer of liposome;

FIG. 11 shows an embodiment drawing of liposome vesicle in 50 ml centrifuge tube;

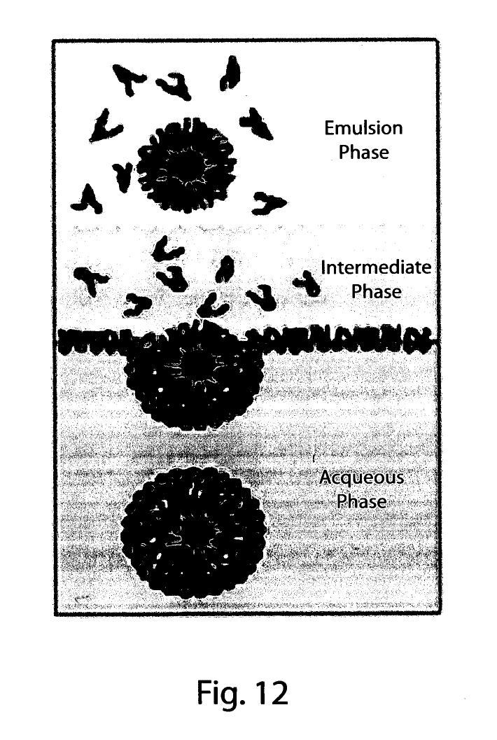

FIG. 12 the assembly drawing of liposomes during the centrifugation step (from “Pautot S, Frisken B J, Weitz FROM. Engineering asymmetric vesicles. Proc. Natl. Acad. Sci. U.S.A. 2003; 100: 10718-21”).

### EXAMPLE 1

### Formation of Biologically Active Asymmetric Liposomes

The asymmetric liposomes are vesicles wherein the lipid component of the external layer is different from the inner one (FIG. 10). In order to obtain asymmetric liposomes it is necessary two different types of lipid suspensions to be prepared: one for external and another for inner monolayer. The inner layer suspension has been prepared in a 100 ml glass bottle. 50 mm of anhydrous dodecane (Sigma Aldrich) and then a phospholipid solution at final lipid concentration of 0.05 mg/ml are added to the bottle. The dodecane and lipid containing bottle is treated in a sonication bath for 30 minutes and left at room temp. for 16-18 hours. On the next morning 250 μl of aqueous solution consisting of 100 mM NaCl and 5 mm Tris buffer are added to the solution containing bottle. The resulting mixture was magnetically mixed for 3 hours obtaining an heterogeneous droplet population (likely surrounded by phospholipids with the hydrophilic head towards the droplet core and the hydrocarbon tail towards the dodecane solution being not polar). For the purposes of the current patent liposomes having phosphatidic acid (PA), as bioactive lipid in the inner layer or phosphatidyl choline (PC), as negative control, are prepared. The suspension for the external layer is prepared in a 50 ml centrifuge tube and consists of 9 parts of anhydrous dodecane and 1 part of silicon oil. To this solution phospholipids in amount suitable to obtain a lipid concentration of 0.05 mg/ml are added. The phospholipids used for the external monolayer for the purpose of this patent are phosphatidyl serine (PS) or phosphatidyl choline (PC).

Four liposome types are produced:
external phosphatidylserine/inner phosphatidic acid (PS/PA);
external phosphatidylserine/inner phosphatidyl choline (PS/PC);
external phosphatidylcholine/inner phosphatidic acid (PC/PA);
phosphatidylcholine both external and inner (PC/PC).

The liposomes are obtained by addition to a 50 ml centrifuge tube in the following order: i) 3 ml of PBS or RPMI 1640 medium, ii) 2 ml of suspension for the external layer, iii) 100 μl of suspension for the inner layer (FIG. 11).

The 50 ml centrifuge tube from 50 ml is centrifuged at 710 rpm×10 minutes, resulting in sedimentation at test tube bottom of spherical mono-layers also present in the phase for inner layer. These monolayer liposomes will cross the lower phase toward the external layer.

Also in the phase for the external layer are present phospholipids equilibrated at the surface bordering with underlying culture medium, with the polar head in PBS (or complete RPMI 1640 medium) and the hydrocarbon tail in dodecane/silicon solution wherein they have been originally depósited, as a monolayer carpet. When the monolayer vesicles, due to centrifugal force, reach this lipid carpet, they continue the downwardly movement pushing against the carpet that passively will surround the monolayer vesicles, finally generating a double-layer vesicle population in the aqueous solution consisting of complete RPMI 1640 medium (FIG. 12). The liposome containing medium is collected using a 5 ml syringe.

### Cytofluorimetric Analysis

The amount of produced liposomes is extrapolated by cytofluorimetry calculating the acquired events in one minute at HIGH acquisition rate (60 μL/min). Being the event amount dependent also on the purity of medium used as collecting aqueous solution, a liposome free sample of single collecting phase (PBS or complete RPMI medium) has been in parallel acquired and the number of the events has been subtracted to the number of the events corresponding to tested vesicle sample. Although this measurement is not highly accurate, the possible quantification error is time limited, whereby it has been used in the calculation of produced vesicles amount. In order to analyse the actual presence of phosphatidylserine outside of bilayers, PS/PA liposomes have been labelled with Annexin V-FITC (Apoptotic Kit—Alexis Biochemicals) according to the supplier instructions. In particular, PS/PA liposomes have been reduced in PBS and centrifuged at 1100 rpm for 5 minutes. After PBS elimination, liposomes have been re-suspended in 390 μL of Binding Buffer (contained in the kit) and 15 μL of Annexin V, and incubated at room temp. in the dark for 10 minutes. After centrifugation at 1100 rpm for 5 minutes, liposomes have been re-suspended in 600 μL of PBS and analysed by cytofluorimetry. The cytofluorimetric analysis has been carried out using a FACScalibur cytofluorimeter (Becton Dickinson Immunocytometry System) equipped with Cell Quest software.

### Fluorescence Confocal Microscopy

In order to detect the presence of PA within obtained liposomes, the latter have been produced using fluorescent PA (λ_{exc}: 460 nm; λₑₘ: 534 nm). Then liposomes have been located on slides previously treated for 30 minutes at 37° C. with poly-L-lisine at 0.1% concentration and analysed by means of Leica fluorescence Laser system confocal microscope in association with Leica inverted microscope (TCS SP2).

### Results

Characterisation of Liposomes

In order to design a system suitable to transport PA directly into target cell, we produced a liposome having PA inside and PS outside, respectively, resulting substantially in two advantages: 1) inner PA is transported into the target cell where it can increase the microbicidal power of the macrophage; 2) the presence of external PS makes the liposome similar to an apoptotic body imparting a natural tropism towards the macrophages, preferential target cells of M. tuberculosis and other intracellular bacterial pathogens. In order to verify the presence of PS outside, the liposomes have been labelled with fluorescent Annexin V (which specifically binds phosphatidylserine in a Ca⁺⁺ dependent mode) and cytofluorimetry analysed. FIG. 1 shows an increase of fluorescence intensity for liposomes containing PS outside compared to not labelled controls. In order to detect the phosphatidic acid presence inside (PA) of produced liposomes, it has been used NBD fluorophore conjugated PA (λₑₓ: 460 nm; λₑₘ: 534 nm) for the composition having inner layer of the phospholipid membrane. The confocal microscopy fluorescence analysis shows the presence of PA inside of liposomes (FIG. 2).

### EXAMPLE 2

### Increased PS/PA Liposome Phagocytosis and Cellular Toxicity Cellular Cultures

In this study monocytic-macrophage THP-1 (American Type Collection Cultures, ATCC) cell line has been used. These cells have been cultured in complete medium (RPMI 1640 containing 10% bovine foetal serum, 5 μg/ml gentamicin and 2 mM L-glutamine) supplemented with 1 mM non essential amino acids and 1 mM sodium pyruvate at 37° C. in humidified atmosphere with 5% CO₂ THP-1 cells have been propagate 2 times a week in 25 cm² or 75 cm² polystyrene flasks and in some experiments cells have been stimulated with 20 ng/ml phorbol 12-miristate 13-acetate (PMA) for 72 hours in order to induce the macrophage differentiation (dTHP-1).

### Fluorescence Microscopy

In order to demonstrate the actual presence of produced liposomes inside of the cell a Delta Vision Applied Precision fluorescence microscope equipped with an Olympus 1×70 microscope, with mercury vapour lamp and Soft Worx acquisition software, has been used. For the purpose to evaluate the liposome internalization in the cells, liposomes having phosphatidylserine outside and fluorescent phosphatidic acid (λ_{exc}: 460 nm; λₑₘ: 534 nm) inside have been prepared. These liposomes have been administered to dTHP-1 cells which were incubated for 90 minutes at 37° C. and 5% CO₂. After cell detachment from the wells using trypsin-EDTA, the latter have been fixed on slides. Finally the internalization of liposomes in the macrophages has been further proved using confocal fluorescence microscopy with Leica fluorescence Laser system confocal microscope in association with Leica inverted microscope (TCS SP2).

### Cell Toxicity

The cell toxicity has been estimated, at time points indicated in FIG. 5, i) analysing the number of viable cells by staining with Trypan Blue (panel A), II) measuring the release of the lactate dehydrogenase (LDH) in culture supernatant (panel B) and iii) by assay with 3-(4,5-dimethyl thiazol-2yl)-2,5 diphenyl tetrazolium bromide (MU) (panel C).

The measurement of LDH release has been carried out using Cyto Tox 96® kit. THP-1 cells have been placed on 48 well plate and differentiation induced with PMA for 72 hours. Subsequently, the cells have been administered to various type vesicles at such concentration to have two vesicle for each cell. The LDH release in supernatant has been monitored after 3 and 5 days after the liposome administration with colorimetric assay using an ELISA reader at 490 nm wavelength.

MTT assay is based on the intracellular reduction of tetrazolium salts, by mitochondrial succinate dehydrogenase enzyme (SDH) as crystals of a bluish product named formazan, which reduction can be realised only in metabolically active cells. To carry out the assay, THP-1 cells have been placed in 96 well plate, at a concentration of 15×10⁴ cells/well and induced to differentiation in the presence of PMA for 3 days. Then cells have been stimulated with 30×10⁴ liposome/well and incubated for 3 hours or 4 days at 37° C. At reported time points 5 mg/ml of MTT diluted in complete culture medium have been added to the culture. After 4 hours of incubation at 37° C., the culture medium was discarded and replaced with 200 μL of dimethyl sulfoxide (DMSO). Sample readings were carried out at 550 nm wavelength using a spectrophotometer. Assay positive control consisted of cells killed by administration of 3% saponin.

### Results

### Phagocytosis Analysis of the Various Liposome Preparations

In order to verify the entrance of liposomes into macrophages, the phagocytosis has been monitored by fluorescence microscopy. Coherently to phagocytic nature of macrophage, we expected that, although this cell phagocytates all vesicle types, it shows a preference towards those cells having outside phosphatidylserine molecules due to similarity thereof to apoptotic bodies. Therefore liposomes having phosphatidylserine outside and fluorescent phosphatidic acid inside, in order to be monitored by fluorescence microscopy. Results obtained using fluorescence microscopy, reported in FIG. 3, show that liposomes consisting of external PS and inner PA are effectively internalized inside of the cells, as it is apparent from the photographs obtained scanning the cells in different sagittal planes, using an optical fluorescence microscope. Results then have been confirmed by confocal fluorescence microscopy (FIG. 4, panel A), where the vesicle (green) appears inside of the cell. After the confirmation of the liposome presence inside of the cell, we compared the phagocytosis extent of two fluorescent liposome preparations containing fluorescent PA inside and PS or PC outside. Results reported in FIG. 4 (panel B) show, as expected, a significant increase of liposome phagocytosis with external PS in comparison with external PC (p<0.001).

### Cell Toxicity

In order to exclude possible cytotoxic effects induced by PS/PA liposomes characterised in having a higher entrance ability in the host cell, we carried out cytotoxicity studies using Trypan Blue staining assay, Lactate dehydrogenase (LDH) release in culture supernatant, formazan crystal formation from MTT. Results shown in FIG. 5 indicate the absence of any significant toxic effect, evaluated in terms of number of viable cells (panel A), LDH enzyme release in the supernatant (panel B) and cell viability (panel C) at kinetic times as indicated in Figure.

### EXAMPLE 3

### PS/PA Liposomes Induce Increase of Intracellular Ca²⁺ Fluorimetric Analysis for the Detection of the Ca⁺⁺ Flow in Monocytes

In order to detect the possible increase of Ca⁺⁺ inside of stimulated cell, THP-1 cells have been incubated in the dark for 1 hour and at 37° C. with Fluo 3 μM (Molecular Probes, NL) fluorophore at 3 μM concentration. The cells have been subsequently washed with PBS and centrifuged at 660 rpm for 5 minutes, re-suspended in RPMI 1640 medium and added to a 96 well plate at a concentration of 105 cell/well. The cells have been stimulated with various types of liposomes at a multiplicity of approximately two liposomes for cell. The determination of Ca⁺⁺ increase has been carried out 20, 40 and 90 minutes after the stimulation. The fluorescence highest value (positive control) has been determined stimulating the cells with 50 ng/ml of phorbol 12-myristate 13-acetate (PMA), meanwhile the minimum fluorescence value has been associated to not treated cells (negative control). Fluorescence has been monitored using a Perkin Elmer LS50B fluorimeter, setting the excitation and emission wavelengths at 505 nm and 530 nm, respectively.

### Results

PS/PA Liposomes Induce an Increase of Intracellular Ca⁺⁺ in Monoliths

From various studies it is apparent that M. tuberculosis have the ability to inhibit Ca⁺⁺ intracellular mobilisation resulting in inhibition of phagolysosome fusion (Malik Z. A et al. 2001). Further PA generation by PLD activity often is associated to Ca⁺⁺ mobilisation. Based on the above, we verified whether PS/PA liposome preparation is suitable to induce, at various kinetic times, an increase of intracellular Ca⁺⁺. Obtained results reported in FIG. 6 show a significant increase of the intracellular Ca⁺⁺ levels, expressed as fluoresce arbitrary units, 20 and 40 minutes after the stimulation with PS/PA preparation but not with other liposome preparations.

### EXAMPLE 4

### PS/PA Liposomes Promote the Phagolysosome Maturation Bacteria

M. tuberculosis H37Rv (MTB) virulent strain was cultured in Middlebrook 7H9 liquid medium, supplemented with 10% ADC (albumin, dextrose, catalase) at 37° C., in humidified atmosphere, with 5% CO₂. After approximately 3 weeks of culture, the bacteria have been washed with PBS (phosphate buffered saline) 0.15 M, pH 7.2 and centrifuged at 21900 rpm for 10 minutes; re-suspended in PBS and sonicated for 3 minutes in order to dissolve aggregates resulting form mycobacterium characteristics. Finally the bacteria have been aliquoted and conserved at −80° C. until the use. Before the freezing, the aliquots have been tittered by serial dilutions and seeded on 60 mm diameter Petri dishes, containing 7H10 agar supplemented with 10% OADC (oleic acid, albumin, dextrose, catalase).

In Vitro Infection Protocol with M. tuberculosis

The bacteria have been thawed at room temp., centrifuged at 9200 rpm for 10 minutes and washed in sterile PBS two times at 9200 rpm for 5 minutes. The bacteria have been at last re-suspended and transferred in a pyrex tube. dTHP-1 cells have been seeded in 48 well plate at a concentration of 3×10⁵ cell/well, and infected at a multiplicity of infection (MOI) of approximately 1 bacterium/cell. After 3 hours 3 washings with warm RPMI 1640 in order to wash the extracellular bacteria. The cells have been then incubated in the presence of various liposomei preparations for various time points as indicated in the different experiments.

### Analysis of the Phagolysosome Maturation by Confocal Microscopy

The analysis of the phagolysosome maturation has been carried out by monitoring the acidification of the phagosome compartment and the expression of protein LAMP-1 in mature phagolysosome, using Lyso Tracker Red (Molecolar Probes, Leiden, NL) acidophilic dye and fluorochrome Alexa fluor 647 (Saint Cruz Biotechnology Inc.) conjugated anti-LAMP-1 antibody. In particular, THP-1 cells (2×10⁶/well) have been seeded in 24 well plate and stimulated with PMA at concentration of 20 ng/ml.

Cells have been incubated with 1:10000 diluted acidophilic Lyso Tracker Red dye for 2 hours at 37° C. with 5% CO₂ in order to label lysosome acid compartments. Then not incorporated dye has been removed with two PBS washings and the cells have been re-suspended in complete medium and infected for 3 hours at 37° C. with M. tuberculosis H37Rv, at multiplicity of infection of 1 bacterium/cell. Then after the removal of the extracellular bacteria present in the supernatant, the cells have been incubated again for 35 minutes at 37° C. with the Lyso Tracker Red dye. After further PBS washing, the cells have been stimulated (liposomes with PS/PA) for 16-18 or absence of 3 mM EGTA. After two PBS washings the cells has been fixed with 4% paraformaldehyde (PFA) and incubated for 20 minutes at 4° C. After an ulterior PBS washing, the cells have been permeabilized by treatment with acetone and methanol (1:1) on ice for 20 minutes. Then two PBS washings have been carried out. In order to visualise the mycobacterium localization, the cells treated with Auramin for 20 minutes at room temp. in the dark, washed and treated with 0.5% acid-alcohol decolorant for 3 minutes at room temp. After a last PBS washing, the cells have been placed on lysine (Sigma) pre-treated slides (VWR International Merck Euro Lab) for 15 minutes at room temp., and left for adhesion purpose at room temp. for 20 minutes. Finally H-1000 Vectashield (Vector Laboratories) medium has been added and the cover slip placed.

### Anti-LAMP-1 Treatment

After the infection arrest by supernatant removal, the cells have been stimulated, fixed and permeabilized according to above protocol. The phagolysosome maturation then has been detected by incubation of dTHP-1 cells with anti-LAMP-1 (Alexa fluor 647) antibody for 1 hour at 4° C. Exceeding antibody has been removed with a PBS washing. The fluorescence has been detected by confocal fluorescence microscope using a combination of argon-crypton (A=488 nm) and helium-neon (A=543 nm) lasers with emission bands at 505-530 nm, 580-600 nm, 600-690 nm, respectively, in order to detect the fluorescence of auramin, Lyso Tracker red and Alexa fluor 647.

### Results

### PS/PA Liposomes Promote the Phagolysosome Maturation

Since the increase of intracellular Ca⁺⁺ levels is a necessary condition for appropriate phagolysosome maturation, the biogenesis of phagolysosome in cells treated with PS/PA in the presence or absence of EGTA, like chelator for extracellular Ca⁺⁺, has been monitored. For this purpose experiments by confocal microscopy using auramin as mycobacterium tracker through binding to mycolic acid of the cellular wall, and Lysotracker Red, acidophilic dye identifying acid compartments of the cell. In FIG. 7 (panel A) it is apparent that, without treatment, mycobacteria reside in not acid compartment acids and do not appear to be green stained. On the contrary, mycobacteria occurring inside of PS/PA liposome treated cells, being located in acid areas (red), appear to be yellow stained. Quantitative analysis depicted in panel B indicates a statistically significant increase of co-localization percentage of mycobacteria in acid phagosomes after stimulation with PS/PA liposomes compared to the control. Moreover, the treatment with an extracellular Ca⁺⁺ chelator (EGTA) inhibits the acidification process of phagosome restoring the initial conditions, proving that PS/PA liposomes promote the phagosome maturation according to a Ca⁺⁺-dependent mode.

Since the phagosome acidification occurs prematurely during t biogenesis thereof and could not be characteristic of mature phagolysosomes, the expression of LAMP (Lysosome Associated Membranes Protein)-1, as late phagolysosome label has been evaluated. FIG. 8 (panel A) shows that the treatment of MTB infected macrophages with PS/PA liposomes promotes the mycobacteria co-localization (green) in LAMP-1 expressing compartments (red), which are yellow stained at fluorescence overlapping. Quantitative analysis depicted in the panel B shows a statistically significant increase of percent co-localization of mycobacteria in LAMP-1 expressing phagosomes after stimulation with PS/PA liposomes compared to the control. Analogously as observed in FIG. 7, the treatment with extracellular Ca⁺⁺ chelator (EGTA) inhibits the PS/PA liposome induced process of phagolysosome maturation.

### EXAMPLE 5

### PS/PA Liposomes Promote the Mycobactericidal Macrophage Response

### UFC Assay

THP-1 cells have been seeded in 24 well plates (5×10⁵ cell/well) and stimulated with PMA at concentration of 20 ng/ml. Differentiated cells (dTHP-1) have been infected with M. tuberculosis H37Rv at a multiplicity of infection of 1 bacterium/cell. 3 hours after the infection, dTHP-1 cells have been washed three times with warm RPMI 1640 medium in order to eliminated extracellular bacteria resulting in the infection arrest. The cells have been then incubated for 3 and 5 days in the presence or absence of different lyposome preparations (PS/PA, PS/PC, PC/PA, PC/PC). The cells have been stimulated with the different liposome types at a multiplicity of about two liposome/cell. At time 0 (3 hours after infection) and after 3 and 5 days, 0.1% saponin (Sigma, St. Louis, Mo.) has been added to each well and incubated at 37° C. for 30 minutes. After this time period, the cell lysates have been picked up, sonicated for 3 minutes and diluted in sterile PBS with addition of 0.01% Tween 80 (Merck, Darmstast, Germany). Then samples have been plated in triplicate on agar Middlebrook 7H10 medium supplemented with 10% OADC and incubated at 37° C., in humidified atmosphere and with 5% CO₂, for 21-24 days. After this period, finally the colony forming units have been detected and counted.

### Results

Inhibition of the Intracellular Increase of M. tuberculosis

Since phagolysosome maturation is associated to the activation of mycobactericidal mechanisms, we evaluated the mycobacterial intracellular increase after stimulation with the following lyposome preparations: i) PS/PA; ii) PS/PC; iii) PC/PA; iv) PC/PC. The mycobacterial increase has been monitored by CFU assays 3 and 5 days after the infection. Results reported in FIG. 9 show the absence of any reduction of the intracellular mycobacterial viability as a result of stimulation with PC/PC liposomes. However, the stimulation with PS/PC or PC/PA liposome preparations induced a significant decrease at the two kinetic steps of mycobacterial viability compared to not stimulated control with further decrease after the treatment with PS/PA liposome preparation.

1. Asymmetric liposomes or aggregates thereof, that are mimetic of apoptotic bodies, comprising:
phosphatidylserine molecules within an outer lipid layer and at least one bioactive lipid involved in antibacterial or antiviral response within an inner lipid layer.

2-45. (canceled)

46. The asymmetric liposomes according to claim 1, wherein said bioactive lipid is selected from the group consisting of phosphatidic acid, lysophosphatidic acid, arachidonic acid, sphingomyelin, sphingosine, sphingosine, 1-phosphate, ceramide, a leukotriene, a prostanoid, a cyclopentenone prostaglandin, and derivatives thereof.

47. The asymmetric liposomes according to claim 1, wherein said bioactive lipid is phosphatidic acid or a derivative thereof.

48. The asymmetric liposomes according to claim 1, wherein said bioactive lipid is a cyclopentenone prostaglandin selected from the group consisting of PGA I, PGA2, PGJ2 and derivatives thereof.

49. The asymmetric liposomes according to claim 1, wherein said inner lipid layer contains phosphatidic acid or a derivative thereof, and cyclopentenone prostaglandin or a derivative thereof, as bioactive lipids within said inner lipid layer.

50. The asymmetric liposomes according to claim 49, wherein said cyclopentenone prostaglandin is selected from PGA1, PGA2, PGJ2 or derivatives thereof.

51. The asymmetric liposomes according to claim 1, which are a carrier system for transferring bioactive lipids to cells suitable to phagocitate apoptotic bodies.

52. The asymmetric liposomes according to claim 46, which are a carrier system for transferring bioactive lipids to cells suitable to phagocitate apoptotic bodies.

53. The asymmetric liposomes according to claim 51, wherein said cells suitable to phagocitate apoptotic bodies are pulmonary cells selected from phagocytes, macrophages, fibroblasts, epithelial cells and endothelial cells.

54. The asymmetric liposomes according to claim 51, wherein said bioactive lipid is phosphatidic acid or a derivate thereof and said cells are macrophages.

55. The asymmetric liposomes according to claim 54, wherein said macrophages are alveolar, alveolar epithelial cells.

56. The asymmetric liposomes according to claim 51, further comprising a cyclopentenone prostaglandin selected from PGA1, PGA2, PGD2 or derivatives thereof.

57. The asymmetric liposomes according to claim 51, wherein said bioactive lipid is a cyclopentenone prostaglandin selected from PGA1, PGA2, PGJ2 or derivatives thereof.

58. The asymmetric liposomes according to claim 51, wherein said cells are pulmonary, dermal or mucosal cells.

59. The asymmetric liposomes according to claim 51, wherein said cells are intestinal cells.

60. The asymmetric liposomes according to claim 58, wherein said pulmonary cells are selected from macrophages, fibroblasts, epithelial cells and endothelial cells.

61. A method for treating pulmonary infections of intracellular bacterial or viral pathogens, comprising:
administering to a patient in need thereof an effective amount of asymmetric liposomes or aggregates thereof that are mimetic of apoptotic bodies, containing phosphatidylserine molecules within an outer lipid layer and at least one bioactive lipid involved in antibacterial or antiviral response within an inner lipid layer.

62. The method of claim 61, wherein said bacterial intracellular pathogens are selected from Mycobacteria sp.; Streptococcus pneumonia: Klebsiella pneumoniae, Pseudomonas aeruginosa; Entobacter sp.; Fusobacterium nucleatum; Bacteroides melaminogenicus; Haemophilus influenzae and Legionella sp.

63. The method of claim 62, wherein said mycobacteria are selected from Mycobacterium tuberculosis, Mycobacterium bovis, Mycobacterium africanum, Mycobacterium avium, Mycobacterium ulcerans, Mycobacterium leprae.

64. The method of claim 61, wherein said viral intracellular pathogens are selected from influenza and parainfluenza virus, respiratory syncytial virus; corona virus; adenovirus and HIV.

65. The method of claim 61, wherein said treatment is for tuberculosis or HIV infection associated tuberculosis.

66. The method of claim 61, further comprising administration of a at least one antibiotic or antiviral.

67. The method of claim 66, wherein said antibiotic is selected from first-line anti-tubercular drugs and second-line anti-tubercular drugs.

68. The method of claim 67, wherein said first line drugs are selected from isoniazid, rifampicin, pyrazinamide, and ethambutol.

69. The method of claim 67, wherein said second line drugs are selected from fluorochinolones, aminoglycosides, cycloserine. macrolides, ethionamide, paraminosalycilic acid, and thiacetazone.

70. A pharmaceutical composition for treating pulmonary infections of intracellular bacterial or viral pathogens, comprising:
one or more asymmetric liposomes or aggregates thereof that are mimetic of apoptotic bodies, containing phosphatidylserine molecules within an outer lipid layer and at least one bioactive lipid involved in antibacterial or antiviral response within an inner lipid layer; and pharmacologically acceptable excipients or adjuvants.

71. The pharmaceutical composition of claim 70, which is suitable for aerial, dermal or mucosal administration.

72. The pharmaceutical composition of claim 70, which is formulated in the form of aerosol.

73. The pharmaceutical composition of claim 70, which is for treatment of pulmonary infections of intracellular bacterial pathogens selected from Mycobacteria sp.; Streptococcus pneumonia: Klebsiella pneumoniae, Pseudomonas aeruginosa; Entobacter sp.; Fusobacterium nucleatum; Bacteroides melaminogenicus; Haemophilus influenzae and Legionella sp. or viral pathogens are selected from influenza and parainfluenza virus, respiratory syncytial virus; corona virus; adenovirus and HIV.

74. The pharmaceutical composition of claim 73, wherein said mycobacteria are selected from Mycobacterium tuberculosis, Mycobacterium bovis, Mycobacterium africanum, Mycobacterium avium, Mycobacterium ulcerans, Mycobacterium leprae.
